SEQUENCE LISTING (12 sequences)

SEQ ID NO: 1 - DNA, 133 bp, wild rat (WT)
ggttccggcactatggcggacccgggggccggcttgcggtgctggctactacagctgcaggagttcgtgtccgcagcagaccgctacaatgctgccggggccagttaccagctgatccgtggcctggggcaag

SEQ ID NO: 2 - DNA, 38 bp, rat
ggttccggcactatggcgggtccgtggcctggggcaag

SEQ ID NO: 3 - DNA, 33 bp, rat
ggttccggcactatgcccgtggcctggggcaag

SEQ ID NO: 4 - DNA, 695 bp, wild rat (WT)
cagttctgagcctcagccgaccaacctcactatgcactataggtatgagaagggggaggggtagtacaggaagaagagaaggtgggttagctgagagagacgggggagcaaaaaagtgggtagccagctcctcaggtaccataccagtttctcatgggataagttatcagttcagaccagatgaagctaggctatgggcagatgtggtacctacctatgtttggcccatcattcttttgccttgtaacccttctctaggtacaagggatctgataataatacattccaggagtgcagccactatctgttctcaaaagagattacttctggctgtcagatacaaaaagaagatatccagctctaccagacatttgttgtccagcttcaggacccccagaaaccccagaggcgagccgaacagaagctaaacctacagaatcttggtaatcgggaaagaagtggccaagaggccagggagcttaaaggcactggagtttatagattgttcttttctcattgttggtcatgggcagaaaggcgaagatggggggggggcggggagggatgaagggaattacctccaagatcctgacttgtctaggccagggcaatgaccacgcacacacatattccagtgatcccatgggctccagagaatctaacactttataacctgagtgaatctcaggtagaac

SEQ ID NO: 5 - DNA, 33 bp, rat
cagttctgagcctcagtgaatctcaggtagaac

SEQ ID NO: 6 - DNA, 783 bp, wild rat (WT)
gcacttggaatagcagttctgagcctcagccgaccaacctcactatgcactataggtatgagaagggggaggggtagtacaggaagaagagaaggtgggttagctgagagagacgggggagcaaaaaagtgggtagccagctcctcaggtaccataccagtttctcatgggataagttatcagttcagaccagatgaagctaggctatgggcagatgtggtacctacctatgtttggcccatcattcttttgccttgtaacccttctctaggtacaagggatctgataataatacattccaggagtgcagccactatctgttctcaaaagagattacttctggctgtcagatacaaaaagaagatatccagctctaccagacatttgttgtccagcttcaggacccccagaaaccccagaggcgagccgaacagaagctaaacctacagaatcttggtaatcgggaaagaagtggccaagaggccagggagcttaaaggcactggagtttatagattgttcttttctcattgttggtcatgggcagaaaggcgaagatggggggggggcggggagggatgaagggaattacctccaagatcctgacttgtctaggccagggcaatgaccacgcacacacatattccagtgatcccatgggctccagagaatctaacactttataacctgagtgaatctcaggtagaactgaggtggaaaagcagatacatagaacgctgtttacaatacttggtgcagtaccggagcaaccgagatcgaagct

SEQ ID NO: 7 - DNA, 40 bp, rat
ccacttggaatagcagatcagaataaccgagatcgaagct

SEQ ID NO: 8 - DNA, 20 bp, Artificial Sequence
  the sequence is synthesized
ttccggcactatggcggacc

SEQ ID NO: 9 - DNA, 20 bp, Artificial Sequence
  the sequence is synthesized
gccagttaccagctgatccg

SEQ ID NO: 10 - DNA, 20 bp, Artificial Sequence
  the sequence is synthesized
cagccgaccaacctcactat

SEQ ID NO: 11 - DNA, 20 bp, Artificial Sequence
  the sequence is synthesized
gagtgaatctcaggtagaac

SEQ ID NO: 12 - DNA, 20 bp, Artificial Sequence
  the sequence is synthesized
gagcaaccgagatcgaagct

(19) United States
(12) Patent Application Publication
(10) Pub. No.: US 2019/0239495 A1
(43) Pub. Date: Aug. 8, 2019

(54) Construction Method for Immunodeficient Rat Model

(71) Applicants: SOUTHERN MEDICAL UNIVERSITY, Guangzhou (CN); GUANGDONG SHENGSAI BIOTECHNOLOGY CO., LTD, Foshan (CN)

(72) Inventor: Yang XU, Guangzhou (CN)

(73) Assignees: SOUTHERN MEDICAL UNIVERSITY, Guangzhou (CN); GUANGDONG SHENGSAI BIOTECHNOLOGY CO., LTD, Foshan (CN)

(21) Appl. No.: 16/342,999

(22) PCT Filed: Mar. 27, 2017
(86) PCT No.: PCT/CN2017/078292

§ 371 Date: Apr. 18, 2019

(30) Foreign Application Priority Data
Feb. 6, 2017 (CN) 201710065515.3

(51) Int. Cl. A01K 67/027

(52) U.S. Cl. CPC A01K 67/0276; A01K 2267/0387; A01K 2227/105; A01K 2217/075

(57) ABSTRACT

A construction method for an immunodeficient rat model includes the following steps. The gRNAs targeted for knocking out a Prkdc gene and an IL2Rγ gene are mixed with CAS9 mRNA respectively, and frozen in RNase-free ultrapure water to obtain injections A and B correspondingly. A human SIRPα genomic DNA is amplified, purified, and frozen in RNase-free ultrapure water to obtain an injection C. The injections A and B are injected into cytoplasm of fertilized eggs of different rats, and the injection C is injected into a pronucleus of a fertilized egg of another rat. The three fertilized eggs are then transplanted into different pseudo-pregnant female rats to breed second-generation rats A, B and C, respectively. The second-generation rats A, B and C are intercrossed to breed an F1 generation; and the F1 generation is further intercrossed to obtain IL2Rγ and Prkdc gene knockout immunodeficient rat models with human SIRPα gene.

### CROSS REFERENCE TO RELATED APPLICATIONS

This application is the national phase entry of International Application No. PCT/CN2017/078292, filed on Mar. 27, 2017, which is based upon and claims priority to Chinese Patent Application No. 201710065515.3, filed on Feb. 6, 2017, the entire contents of which are incorporated herein by reference.

### TECHNICAL FIELD

The embodiments of the present invention relate to the field of rat model construction, particularly to a construction method for an immunodeficient rat model.

### BACKGROUND

Animal models are mainly used in the study of experimental physiology, experimental pathology, and experimental therapeutics (including the screening of new drugs). The development of human diseases is very complicated, especially since studies exploring disease mechanisms using humans as the test subjects are slow. Therefore, clinical trials and experimentation is limited not only in the amount of studies and length of studies, but also restricted by moral implications. Thus, animal models are primarily used; and with the physiologically relevant research using animal models, it is possible to intentionally change the conditions that are impossible or difficult to exclude under natural conditions. In this way, the experimental results of the model can be more accurately observed and configured for comparative research with human diseases, which is conducive to a more convenient and effective research on the mechanisms of the pathogenesis of human diseases and the prevention and control measures.

Immunodeficient animal models can be used in the drug development, transplantation studies, and the study of the therapeutic mechanisms of human diseases. At present, the most commonly used immunodeficient animal models are rodents, specifically mice. Since the homology between rats and humans is relatively closer than humans and mice, the use of rat models to study related diseases of human beings has obvious advantages over mouse models. To measure the degree of immunodeficiency, the proportions of T lymphocytes, B lymphocytes and NK cells in the body are mainly measured. Japanese scholar Tomoji et al. (Mashimo T, Takizawa A, et al: Generation and characterization of severe combined immunodeficiency rats. Cell reports 2012, 2:685-694, Mashimo T, Takizawa A, et al: Generation of knockout rats with X-linked severe combined immunodeficiency (X-SCID) using zinc-finger nucleases. PloS one 2010, 5:e8870) used the Zinc-Finger nuclease technique to generate IL2Rγ and Prkdc gene knockout rats, and confirmed that, in IL-2Rγ homozygous knockout rats, T cell numbers in peripheral blood, bone marrow and spleen were significantly reduced, B cells and NK cells disappeared in peripheral blood and bone marrow, and only some residues of B cells and NK cells can be found in the spleen; in Prkdc gene knockout rats, CD4⁻CD8⁺, CD4⁺CD8⁻ and CD4⁺CD8⁺ T cells all disappeared, B cells also significantly disappeared, but the number of NK cells was increased; in double-knockout Prkdc and IL2Rγ rats, T lymphocytes, B lymphocytes and NK cells all disappeared. However, the inventors have found that the immunodeficient rat models of the prior art have at least the following problem: when human CD34⁺ hematopoietic stem cells were transplanted into double-knockout Prkdc and IL2Rγ rat models, they cannot engraft and no human immune cells were generated.

Therefore, the existing immunodeficient rat models cannot meet the needs of drug testing and xenogeneic cell transplantation research, etc., which is an urgent problem to be solved by those skilled in the art.

### SUMMARY

In order to solve the above technical problems, the embodiment of the present invention provides a new immunodeficient rat model, which fills the strain vacancies of the immunodeficient rat model and can meet the requirements of drug testing and xenogeneic cell transplantation research, etc.

In a first aspect, a construction method for an immunodeficient rat model is provided, including the following steps:
(1) the gRNAs targeted for knocking out the Prkdc gene and IL2Rγ gene of rat DNA were separately transcribed in-vitro, then mixed with the in-vitro transcribed CAS9 mRNA, and frozen in RNase-free ultrapure water to obtain an injection A and an injection B correspondingly;
(2) the human SIRPα genomic DNA was amplified, purified, and frozen in RNase-free ultrapure water to obtain an injection C;
(3) the injection A and the injection B were injected into the cytoplasm of the fertilized eggs of different rats by the microinjection method, and then the fertilized eggs of rats were respectively transplanted into different pseudo-pregnant female rats, and a second-generation rat A and a second-generation rat B were bred;
(4) the injection C was injected into the pronucleus of the fertilized egg of rat by the microinjection method, and then the fertilized egg of rat was transplanted into the pseudo-pregnant female rat to obtain a second-generation rat C; and
(5) the second-generation rat A, the second-generation rat B and the second-generation rat C were intercrossed to generate the F1 generation; the F1 generation was further intercrossed to obtain IL2Rγ and Prkdc gene knockout immunodeficient rat models with human SIRPα gene.

In combination with the first aspect, in one possible implementation modes of the first aspect, two gRNA sequences are used for knocking out rat Prkdc gene in step (1), which are respectively: the first exon gRNA sequence 1: TTCCGGCACTATGGCGGACC; the first exon gRNA sequence 2: GCCAGTTACCAGCTGATCCG.

In combination with the first aspect or some of the above possible implementation modes, in one possible implementation mode of the first aspect, two gRNA sequences are used for knocking out rat IL2Rγ gene in step (1), which are respectively: the second exon gRNA sequence 1: CAGCCGACCAACCTCACTAT; the fourth exon gRNA sequence 2: GAGTGAATCTCAGGTAGAAC.

In combination with the first aspect or some of the above possible implementation modes, in one possible implementation mode of the first aspect, two other gRNA sequences are further used for knocking out the rat IL2Rγ gene, which are respectively: the second exon gRNA sequence 1: CAGCCGACCAACCTCACTAT; the fourth exon gRNA sequence 3: GAGCAACCGAGATCGAAGCT.

In combination with the first aspect or some of the above possible implementation modes, in one possible implementation mode of the first aspect, the in-vitro transcription of the CAS9 mRNA is carried out by using a T7 transcription kit, a T3 transcription kit, or an SP6 transcription kit.

In combination with the first aspect or some of the above possible implementation modes, in one possible implementation mode of the first aspect, the pseudo-pregnant female rats in step (3) are raised under specific pathogen-free (SPF) conditions.

In combination with the first aspect or some of the above possible implementation modes, in one possible implementation mode of the first aspect, the rat fertilized eggs in step (3) were taken from pregnant female rats raised under the SPF conditions.

In a second aspect, an immunodeficient rat model is provided, the rat model is an IL2Rγ and Prkdc gene knockout rat with human SIRPα gene.

In combination with the second aspect, in one possible implementation mode of the second aspect, the immunodeficient rat model is constructed by any of the methods of the first aspect.

In the above technical solutions, the human SIRPα (hSIRPα) gene is overexpressed in the IL2Rγ and Prkdc knockout rat, and then a rat model without immune function and overexpressing the human SIRPα gene is constructed, thereby obtaining a rat model that has no immunization effect on xenografts, so that when this rat model is used for xeno-transplantation, besides T lymphocytes, B lymphocytes and NK cells, other immune cells also not attack human-derived xenografts, meeting the needs of drug testing, xenogeneic cell transplantation study, and other related studies. The immunodeficient rat model obtained by this method (1) fills the strain vacancies of the immunodeficient rat model, (2) has compliance with the experimental indicators, (3) allows human cells to be implanted efficiently, and (4) has a pure strain background.

### BRIEF DESCRIPTION OF THE DRAWINGS

FIG. 1 is a schematic diagram showing a position of a gRNA used for knocking out a Prkdc gene in rat DNA in one of the specific implementation modes of an immunodeficient rat model construction method provided by the present invention.

FIG. 2 is a schematic diagram showing a position of a gRNA used for knocking out an IL2Rγ gene in rat DNA in one of the specific implementation modes of an immunodeficient rat model construction method provided by the present invention.

FIG. 3 is a flow cytometric data of the expression of human SIRPα (hSIRPα) and rat SIRPα (rSIRPα) in peripheral blood of a normal wild type rat (WT) and a transgenic rat overexpressing hSIRPα in one of the specific implementation modes of the present invention.

FIG. 4 is a flow cytometric map of NK cells, B lymphocytes and T lymphocytes in peripheral blood of a normal wild type rat (WT) and a heterozygous knockout Prkdc (Prkdc^{+/-}) rat and a double-knockout Prkdc (Prkdc^{-/-}) rat in one of the specific implementation modes of the present invention.

FIG. 5 is a flow cytometric map of NK cells, B lymphocytes and T lymphocytes in peripheral blood of a normal wild type rat (WT) and a heterozygous knockout IL2Rγ (IL2Rγ^{+/-}) rat and a double-knockout IL2Rγ (IL2Rγ^{-/-}) rat in one of the specific implementation modes of the present invention.

FIG. 6 is a flow cytometric data of NK cells, B lymphocytes and T lymphocytes in peripheral blood of a normal wild type rat (WT) and a SG (IL2Rγ^{-/-}, Prkdc^{-/-}) rat model and an NSG (hSIRPα⁺, IL2Rγ^{-/-}, Prkdc^{-/-}) rat model, and expressions of hSIRPα and rSIRPα in one of the specific implementation modes of the present invention.

FIG. 7 is a diagram showing detection results of expressions of human immune cells in peripheral blood and bone marrow after 5 weeks of a tail vein injection of human hematopoietic stem cells into an immunodeficient rat model constructed by one implementation mode of the present invention.

FIG. 8 is a diagram showing tumor formation situations after subcutaneous transplantations of tumor cells and embryonic stem cells in an SG (IL2Rγ^{-/-}, Prkdc^{-/-}) rat model and an NSG (hSIRPα⁺, IL2Rγ^{-/-}, Prkdc^{-/-}) rat model.

### DETAILED DESCRIPTION OF THE EMBODIMENTS

In order to expound the technical solutions of the present invention more clearly, the technical solutions of the present invention will be further described in detail below with reference to the specific implementation modes.

The inventors have found that the immunodeficient rat models of the prior art have at least the following problem: human CD34⁺ hematopoietic stem cells were transplanted into double-knockout Prkdc and IL2Rγ rat models, but no human immune system cells were generated. The inventors have analyzed and suggested that the main possible reason was the failure of transplantation caused by the xenogeneic rejection of macrophages and monocytes in the rat model.

Therefore, a construction method for an immunodeficient rat model is provided in a specific implementation mode, including the following steps: (1) gRNAs targeted for knocking out the Prkdc gene and IL2Rγ gene of rat DNA were separately transcribed in-vitro, then mixed with the in-vitro transcribed CAS9 mRNA, and frozen in RNase-free ultrapure water to obtain the corresponding injection A and injection B;
(2) a human SIRPα genomic DNA was amplified, purified, and frozen in RNase-free ultrapure water to obtain an injection C;
(3) the injection A and the injection B were injected into the cytoplasm of the fertilized eggs of different rats by the microinjection method, and then the fertilized eggs of rats were transplanted into different pseudo-pregnant female rats, and a second-generation rat A and a second-generation rat B were bred;
(4) the injection C was injected into the pronucleus of the fertilized egg of rat by the microinjection method, and then the fertilized egg of rat was transplanted into the pseudo-pregnant female rat to obtain a second-generation rat C; and
(5) the second-generation rat A, the second-generation rat B and the second-generation rat C were intercrossed to generate the F1 generation; the F1 generation was further intercrossed to obtain IL2Rγ and Prkdc gene knockout immunodeficient rat models with human SIRPα gene.

The above-mentioned microinjection method refers to direct injection of a fragment of an exogenous gene into a prokaryotic embryo or a cultured cell by using a glass microinjection needle having a very fine tip (0.1 to 0.5 μm), and then the exogenous gene is embedded in the chromosome of the host by means of the phenomena of recombination, deletion, replication or translocation that may occur in the host genome sequence.

The above-mentioned human SIRPα genomic DNA, i.e., Signal Regulatory Protein Alpha genomic DNA, can be purchased directly through commercial vendors. For example, the human SIRPα genomic DNA used in the above solution was purchased from Life Technologies (BAC, RP11-993C19). The originally purchased human SIRPα genomic DNA needs to be amplified and purified first, because the microinjection of the fertilized egg requires a sufficiently pure injection, otherwise it will easily cause the fertilized egg to fail to develop properly, thus no positive result will be obtained.

In the above technical solution, SIRPα is mainly expressed on the surfaces of monocytes and macrophages, and bound to the ligand of CD47 to mediate a negative regulatory signal, i.e., a “don't-eat-me” signal. Through the creative work of the inventors and a lot of experiments, the gRNAs targeted for knocking out the Prkdc gene and IL2Rγ gene of rat DNA were separately transcribed in-vitro, then mixed with the in-vitro transcribed CAS9 mRNA in proportion, and separately injected into the cytoplasm or pronucleus of different fertilized eggs of fertilized female rat by the microinjection method. In the fertilized egg, the Cas9 mRNA was translated into a protein, i.e., Cas9 protein, relying on the eukaryotic translation system. The Cas9 protein was bound to the gRNA, and then bound to the targets through the mediation of the gRNA. The Cas9 protein cleaved the target, and destroyed the immune-related gene sequence to silence the expression of the immune-related gene. Subsequently, the fertilized eggs were transplanted into the pseudo-pregnant female rat to breed the second-generation rat A that knocked out the Prkdc gene and the second-generation rat B that knocked out the IL2Rγ gene. The human SIRPα genomic DNA was injected into the pronucleus of the fertilized egg of another rat by the microinjection method, the SIRPα genomic DNA was randomly inserted into the genome during the division of the fertilized egg, thereby breeding the second-generation rat C expressing the human SIRPα. Finally, the second-generation rat A, the second-generation rat B and the second-generation rat C were intercrossed to breed the F1 generation, and then the F1 generation was intercrossed to establish a stable homozygous strain of the Prkdc and IL2Rγ gene knockout immunodeficient rat with stable expression of the human SIRPa (hSIRPa) gene, thereby constructing a rat model having no immune function and overexpressing human SIRPα gene.

The rat model obtained by the above-mentioned method has no immune effect on xenogeneic transplantation, so that when this rat model is used for xeno-transplantation, besides T lymphocytes, B lymphocytes and NK cells, other immune cells also not attack human-derived xenografts, meeting the needs of drug testing, xenogeneic cell transplantation study, and other related studies. The immunodeficient rat model is a good platform for humanized rat. By transplanting human ES cells and tumor cells in rats, the results showed good xenograft ability. After transplantation of human CD34⁺ hematopoietic stem cells, a humanized hematopoiesis system rat model can be established. In addition, the immunodeficient rat model can also be applied to research fields such as human stem cell transplantation, tumor biology, reconstruction of humanized immune system, human antibody manufacturing, and HIV research.

The immunodeficient rat model established by the above-mentioned technical solution not only fills the strain vacancies of the immunodeficient rat model, compliance with the experimental indicators, but also has a pure strain background.

The above-mentioned second-generation rat A with knocked out the Prkdc gene and the second-generation rat B with knocked out the IL2Rγ gene were both constructed using the CRISPR/Cas9 technology. The CRISPR/Cas9 technology and previous Zinc-Finger and TALEN technologies can achieve gene knockout, but because each technology has certain requirements on gene editing sites, the resulting rat DNA knockout sequences (sites) are different. In addition, the CRISPR/Cas9 technology is simple in design, convenient in operation and has higher efficiency.

The above-mentioned gRNA, also known as guide RNA, is one of the core parts of the CRISPR/Cas9 technology.

Further, two gRNA sequences are used for knocking out the rat Prkdc gene in the step (1), which are respectively: the first exon gRNA sequence 1: TTCCGGCACTATGGCGGACC; the first exon gRNA sequence 2: GCCAGTTACCAGCTGATCCG.

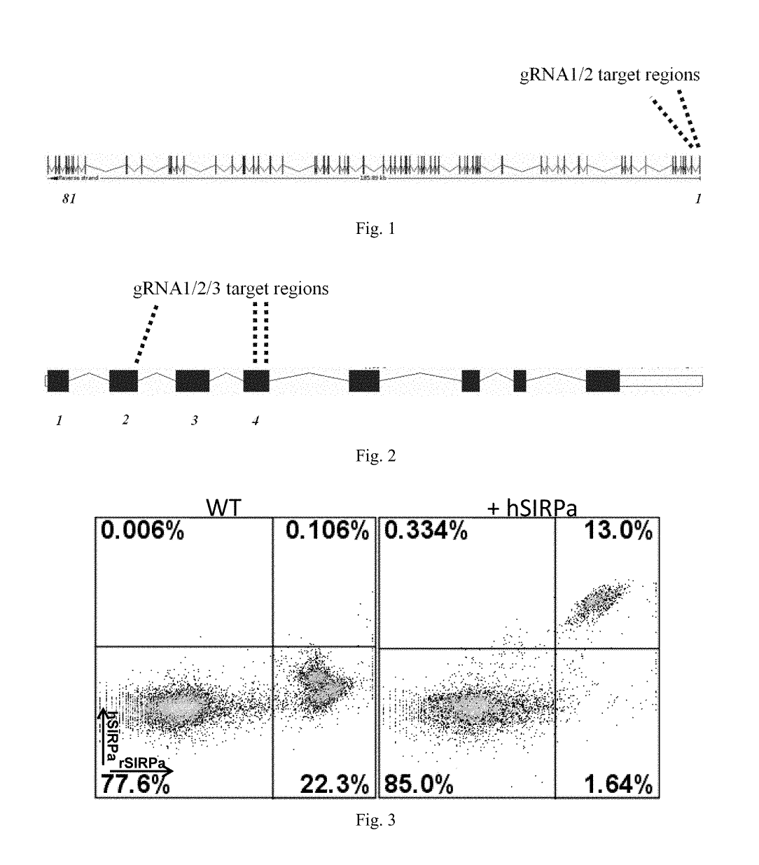

In reference with FIG. 1, an approximate position guided by a gRNA used for knocking out the Prkdc gene of the rat DNA in the first exon is shown.

The sequence of the Prkdc gene of wild rat is:

### SD-Prkdc-WT#1:

GGTTCCGGCACTATGGCGGACCCGGGGGCCGGCTTGCGGTGCTGGCTACT
ACAGCTGCAGGAGTTCGTGTCCGCAGCAGACCGCTACAATGCTGCCGGGG
CCAGTTACCAGCTGATCCGTGGCCTGGGGCAAG.

The gene was edited by the CRISPR/Cas9 technology. Cas9 binds to the target DNA sequence under the guidance of gRNA. The Cas9 protein cleaves the target DNA sequence at the third base of the protospacer adjacent motif (PAM) sequence, and the broken DNA will undergo an end-joining repair, some bases may be inserted during the process. In this solution, two Prktd knockout sequences are obtained after knocking out the Prktd gene by the CRISPR/Cas9 technology.

In the first Prkdc knockout sequence, a total of 95 bases from the 20^{th} to the 114^{th} were deleted, and 1 base mutated, i.e., the 115^{th} base mutated from A to G. The sequence is specifically as follows:

### Deletion #1:

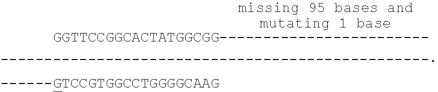

In the second Prkdc knockout sequence, a total of 100 bases from the 16^{th} to the 115^{th} were deleted, and 1 base mutated, i.e., the 116^{th} base mutated from T to C. The sequence is specifically as follows:

### Deletion #2:

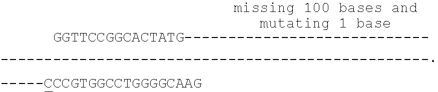

Further, two gRNA sequences are used for knocking out the rat IL2Rγ gene in the step (1), which are respectively: the second exon gRNA sequence 1: CAGCCGACCAACCTCACTAT; the fourth exon gRNA sequence 2: GAGTGAATCTCAGGTAGAAC.

Further, two other gRNA sequences are further used for knocking out the rat IL2Rγ gene in the step (1), which are respectively: the second exon gRNA sequence 1: CAGCCGACCAACCTCACTAT; the fourth exon gRNA sequence 3: GAGCAACCGAGATCGAAGCT.

In reference with FIG. 2, the approximate positions guided by three different gRNAs used for knocking out the IL2Rγ gene of rat DNA in the first exon are shown.

The sequence 1 of the IL2Rγ_gene of wild rat is:

### SD-IL2Rγ-WT#2:

CAGTTCTGAGCCTCAGCCGACCAACCTCACTATGCACTATAGGTATGAGA
AGGGGGAGGGGTAGTACAGGAAGAAGAGAAGGTGGGTTAGCTGAGAGAGA
CGGGGGAGCAAAAAAGTGGGTAGCCAGCTCCTCAGGTACCATACCAGTTT
CTCATGGGATAAGTTATCAGTTCAGACCAGATGAAGCTAGGCTATGGGCA
GATGTGGTACCTACCTATGTTTGGCCCATCATTCTTTTGCCTTGTAACCC
TTCTCTAGGTACAAGGGATCTGATAATAATACATTCCAGGAGTGCAGCCA
CTATCTGTTCTCAAAAGAGATTACTTCTGGCTGTCAGATACAAAAAGAAG
ATATCCAGCTCTACCAGACATTTGTTGTCCAGCTTCAGGACCCCCAGAAA
CCCCAGAGGCGAGCCGAACAGAAGCTAAACCTACAGAATCTTGGTAATCG
GGAAAGAAGTGGCCAAGAGGCCAGGGAGCTTAAAGGCACTGGAGTTTATA
GATTGTTCTTTTCTCATTGTTGGTCATGGGCAGAAAGGCGAAGATGGGGG
GGGGGCGGGGAGGGATGAAGGGAATTACCTCCAAGATCCTGACTTGTCTA
GGCCAGGGCAATGACCACGCACACACATATTCCAGTGATCCCATGGGCTC
CAGAGAATCTAACACTTTATAACCTGAGTGAATCTCAGGTAGAAC.

Using the CRISPR/Cas9 technology, and the second exon gRNA sequence 1 and the fourth exon gRNA sequence 2 as the guide RNAs, the first IL2Rγ knockout sequence was obtained, in which a total of 662 bases from the 17^{th} to the 678^{th} were deleted. The sequence is specifically as follows:

### Deletion #3:

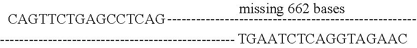

The sequence 2 of the IL2Rγ gene of wild rat is:

### SD-IL2Rγ-WT#3:

GCACTTGGAATAGCAGTTCTGAGCCTCAGCCGACCAACCTCACTATGCACT
ATAGGTATGAGAAGGGGGAGGGGTAGTACAGGAAGAAGAGAAGGTGGGTTA
GCTGAGAGAGACGGGGGAGCAAAAAAGTGGGTAGCCAGCTCCTCAGGTACC
ATACCAGTTTCTCATGGGATAAGTTATCAGTTCAGACCAGATGAAGCTAGG
CTATGGGCAGATGTGGTACCTACCTATGTTTGGCCCATCATTCTTTTGCCT
TGTAACCCTTCTCTAGGTACAAGGGATCTGATAATAATACATTCCAGGAGT
GCAGCCACTATCTGTTCTCAAAAGAGATTACTTCTGGCTGTCAGATACAAA
AAGAAGATATCCAGCTCTACCAGACATTTGTTGTCCAGCTTCAGGACCCCC
AGAAACCCCAGAGGCGAGCCGAACAGAAGCTAAACCTACAGAATCTTGGTA
ATCGGGAAAGAAGTGGCCAAGAGGCCAGGGAGCTTAAAGGCACTGGAGTTT
ATAGATTGTTCTTTTCTCATTGTTGGTCATGGGCAGAAAGGCGAAGATGGG
GGGGGGGCGGGGAGGGATGAAGGGAATTACCTCCAAGATCCTGACTTGTCT
AGGCCAGGGCAATGACCACGCACACACATATTCCAGTGATCCCATGGGCTC
CAGAGAATCTAACACTTTATAACCTGAGTGAATCTCAGGTAGAACTGAGGT
GGAAAAGCAGATACATAGAACGCTGTTTACAATACTTGGTGCAGTACCGGA
GCAACCGAGATCGAAGCT.

Using the CRISPR/Cas9 technology, and the second exon gRNA sequence 1 and the fourth exon gRNA sequence 3 as the guide RNAs, the second IL2Rγ knockout sequence was obtained, in which a total of 751 bases from the 17^{th} to the 767^{th} were deleted, and a total of 8 bases from 17^{th} to the 27^{th} were inserted after the 16^{th} base. The sequence is specifically as follows:

### Deletion #4:

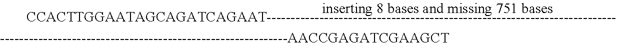

Further, in the step (1), the in-vitro transcription of the CAS9 mRNA is carried out by using a T7 transcription kit, a T3 transcription kit or an SP6 transcription kit. The selection of the transcription kit is determined by the promoter that initiates the gRNA and Cas9 proteins.

Further, the pseudo-pregnant female rats in step (3) and step (4) are raised under SPF conditions.

The SPF (Specific pathogen Free) grade refers to the grade of no specific pathogen. The purpose of this solution is to construct immunodeficient rats, and the homozygous immunodeficient rats may be generated in the first generation by using the CRISPR/Cas9 knockout. Therefore, the preferred pseudo-pregnant female rats need to be raised under the SPF conditions.

In the technical solution of the present invention, the numbers of steps (1), (2), (3), (4) and (5) are only for the convenience of description, and are not used to limit the order of various steps in the construction method. As long as it is logically reasonable, the order of various steps in the method can vary. For example, the above step (1) can be performed independently with step (2) at the same time; step (2) can be performed before step (1); also, step (3) and step (4) can be performed independently at the same time, and there is no specific order limitation between the two steps.

The above-described specific implementation modes will be further described below through the following embodiments, but the present invention is not intended to be limited within the scope of the embodiments. The experimental methods in the following embodiments without specifying the specific conditions are selected according to conventional methods and conditions, or according to the instructions of the products. Other reagents, materials, and equipment that are not specifically described can be purchased directly through commercial channels.

### Embodiment 1

(1) The gRNAs targeted for knocking out the Prkdc gene and IL2Rγ gene of rat DNA were separately transcribed in-vitro, then mixed with the in-vitro transcribed CAS9 mRNA, and frozen in RNase-free ultrapure water to obtain the corresponding injection A and injection B for standby application.

Two gRNA sequences were used for knocking out the rat Prkdc gene, which were respectively: the first exon gRNA sequence 1: TTCCGGCACTATGGCGGACC; the first exon gRNA sequence 2: GCCAGTTACCAGCTGATCCG.

Two gRNA sequences were used for knocking out the rat IL2Rγ gene, which were respectively: the second exon gRNA sequence 1: CAGCCGACCAACCTCACTAT; the fourth exon gRNA sequence 2: GAGTGAATCTCAGGTAGAAC.

The in-vitro transcription of the CAS9 mRNA was carried out by using a T7 transcription kit.

(2) The human SIRPα genomic DNA was amplified, purified, and frozen in RNase-free ultrapure water to obtain injection C for standby application.

(3) The fallopian tubes were obtained from the peritoneal cavities of the pregnant female rats raised under SPF conditions, washed with physiological saline at a concentration of 0.9%, and the fertilized eggs were observed, picked and collected under a microscope.

(4) The injection A and the injection B were injected into the cytoplasm of the different fertilized eggs obtained in the step (3) by the microinjection method, and then the fertilized eggs of rats were transplanted into different pseudo-pregnant female rats raised under the SPF conditions, and a second-generation rat A and a second-generation rat B were bred;

(5) The injection C was injected into the pronucleus of another fertilized egg obtained in the step (3) by the microinjection method, and then the fertilized egg of rat was transplanted into another pseudo-pregnant female rat raised under the SPF conditions to obtain a second-generation rat C; and

(6) The second-generation rat A, the second-generation rat B and the second-generation rat C were intercrossed to breed an F1 generation; the F1 generation was further intercrossed to obtain IL2Rγ and Prkdc gene knockout immunodeficient rat models with human SIRPα gene.

### Embodiment 2

(1) The gRNAs targeted for knocking out the Prkdc gene and IL2Rγ gene of rat DNA were separately transcribed in-vitro, then mixed with the in-vitro transcribed CAS9 mRNA, and frozen in RNase-free ultrapure water to obtain the corresponding injection A and injection B for standby application.

Two gRNA sequences were used for knocking out the rat Prkdc gene, which were respectively: the first exon gRNA sequence 1: TTCCGGCACTATGGCGGACC; the first exon gRNA sequence 2: GCCAGTTACCAGCTGATCCG.

Two gRNA sequences were used for knocking out the rat IL2Rγ gene, which were respectively: the second exon gRNA sequence 1: CAGCCGACCAACCTCACTAT; the fourth exon gRNA sequence 2: GAGTGAATCTCAGGTAGAAC.

Two other gRNA sequences were used for knocking out the rat IL2Rγ gene, which were respectively: the second exon gRNA sequence 1: CAGCCGACCAACCTCACTAT; the fourth exon gRNA sequence 3: GAGCAACCGAGATCGAAGCT.

The in-vitro transcription of the CAS9 mRNA was carried out by using a T7 transcription kit.

(2) The human SIRPα genomic DNA was amplified, purified, and frozen in RNase-free ultrapure water to obtain injection C for standby application.

(3) The fallopian tubes were obtained from the peritoneal cavities of the pregnant female rats raised under the SPF conditions, washed with physiological saline at a concentration of 0.9%, and the fertilized eggs were observed, picked and collected under a microscope.

(4) The injection A and the injection B were injected into the cytoplasm of the different fertilized eggs obtained in the step (3) by the microinjection method, and then the fertilized eggs of rats were transplanted into different pseudo-pregnant female rats raised under the SPF conditions, and a second-generation rat A and a second-generation rat B were bred;

(5) The injection C was injected into the pronucleus of another fertilized egg obtained in the step (3) by the microinjection method, and then the fertilized egg of rat was transplanted into another pseudo-pregnant female rat raised under the SPF conditions to obtain a second-generation rat C; and

(6) The second-generation rat A, the second-generation rat B and the second-generation rat C were intercrossed to breed an F1 generation; the F1 generation was further intercrossed to obtain IL2Rγ and Prkdc gene knockout immunodeficient rat models with human SIRPα gene.

FIG. 3 is a flow cytometric map of expressions of human SIRPα (hSIRPα) and rat SIRPα (rSIRPα) in peripheral blood of a normal wild rat (WT) and a rat overexpressing hSIRPα (i.e., a second-generation rat C) in an embodiment of the present invention.

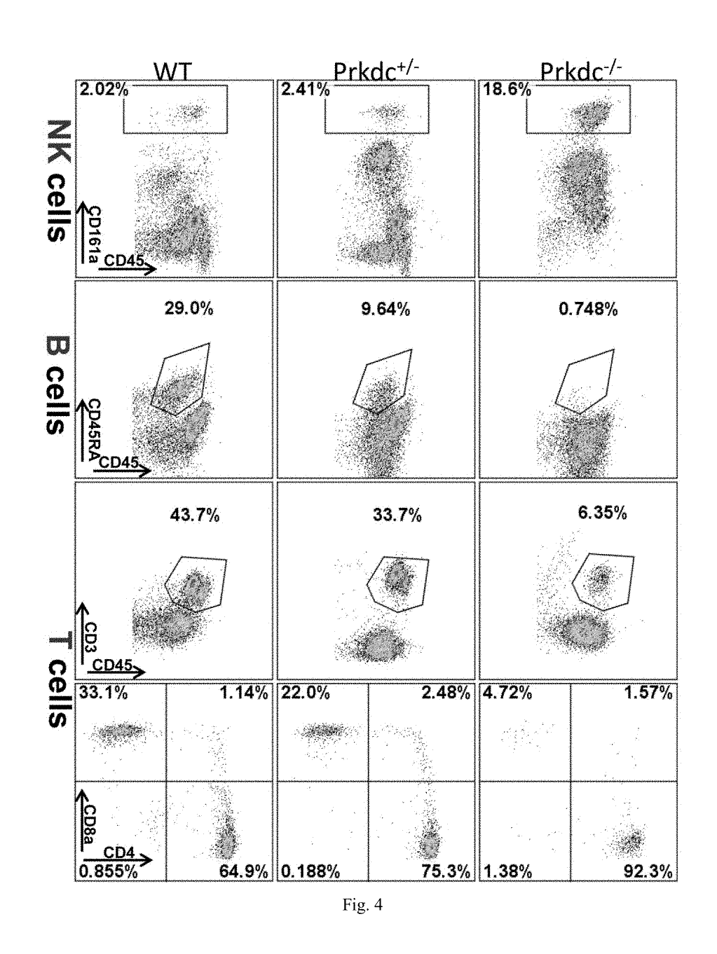

FIG. 4 is a flow cytometric map of NK cells, B lymphocytes and T lymphocytes in peripheral blood of a normal wild rat (WT) and a single-knockout Prkdc (Prkdc^{+/-}) rat and a double-knockout Prkdc (Prkdc^{-/-}) rat.

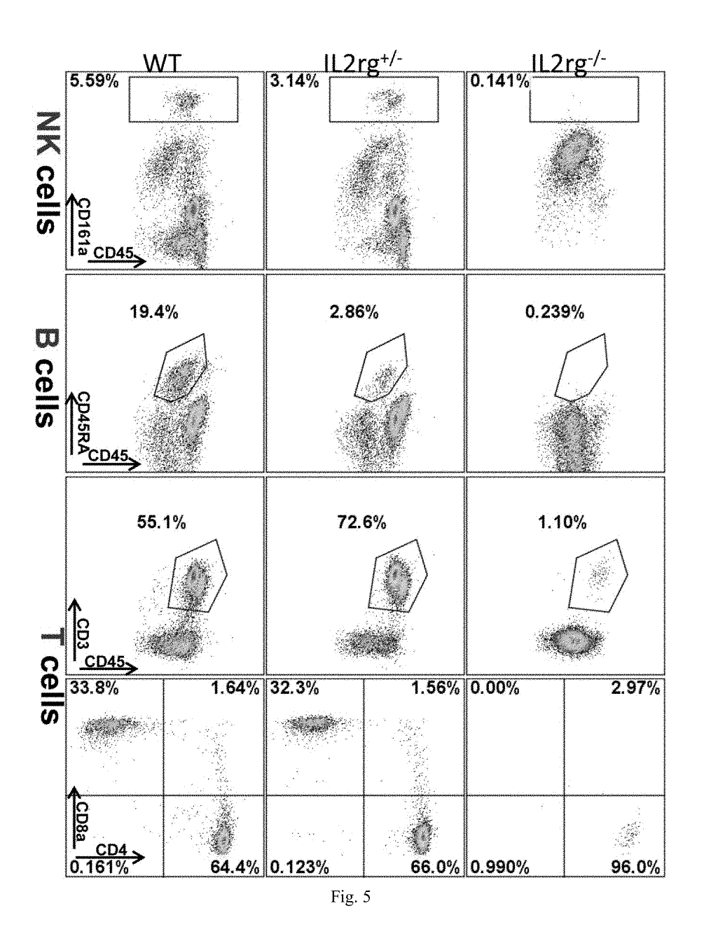

FIG. 5 is a flow cytometric map of NK cells, B lymphocytes and T lymphocytes in peripheral blood of a normal wild rat (WT) and a single-knockout IL2Rγ (IL2Rγ^{+/-}) rat and a double-knockout IL2Rγ (IL2Rγ^{-/-}) rats.

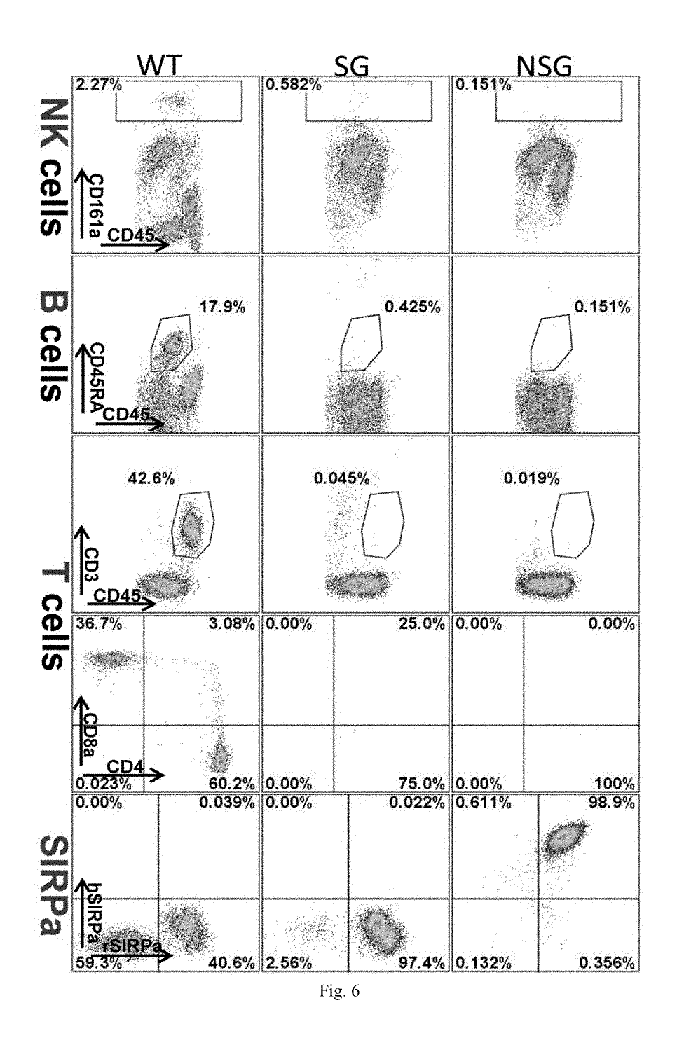

FIG. 6 is a flow cytometric map of NK cells, B lymphocytes and T lymphocytes in peripheral blood of a normal wild rat (WT) and an SG (IL2Rγ^{-/-}, Prkdc^{-/-}) rat model and an NSG (hSIRPα, Prkdc^{-/-}) rat model, and expressions of hSIRPα and rSIRPα.

In FIG. 4, the single-knockout Prkdc (Prkdc^{+/-}) rats and the double-knockout Prkdc (Prkdc^{-/-}) rats are two possible knockout examples of the second-generation rat A. In FIG. 5, the single-knockout IL2Rγ (IL2Rγ^{+/-}) rats and the double-knockout IL2Rγ (IL2Rγ^{-/-}) rats are two possible knockout examples of the second-generation rat B. In FIG. 6, the NSG rat is an immunodeficient rat model constructed by a preferred embodiment of the present invention.

### Comparative Example 1

NSG rat was used to perform human CD34⁺ hematopoietic stem cell transplantation through tail vein injection, and 1*10⁶ human CD34⁺ cells were transplanted. After 5 weeks, human immune cells were detected in both peripheral blood and bone marrow, without transplanting human thymus, human B cells (hCD19 positive) dominate, accounting for more than 65%, while T cells (hCD3 positive) and monocytes/macrophages (hCD14 positive) account for a small proportion. This fully indicates that NSG rats can be used to establish humanized rats.

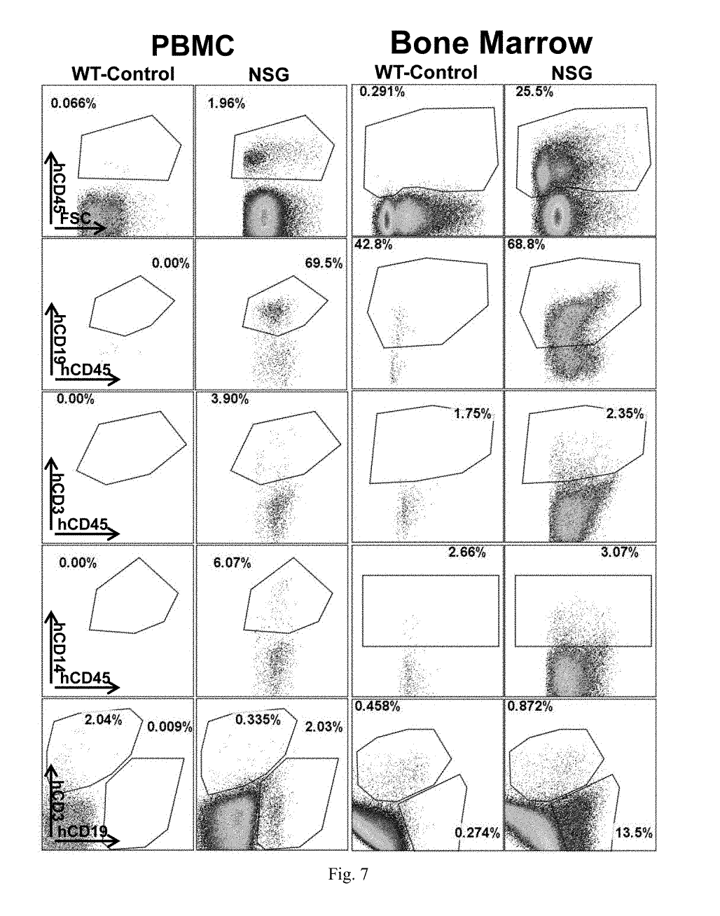

FIG. 7 is a diagram showing a development situation of human peripheral blood cells after 5 weeks of transplantation of human CD34⁺ hematopoietic stem cell through a tail vein injection into an immunodeficient rat model constructed by one embodiment of the present invention.

### Comparative Example 2

Lung cancer tumor cells H460 and human embryonic stem cells H9 were subcutaneously injected into SG and NSG rat models, with 1*10⁵ cells, respectively.

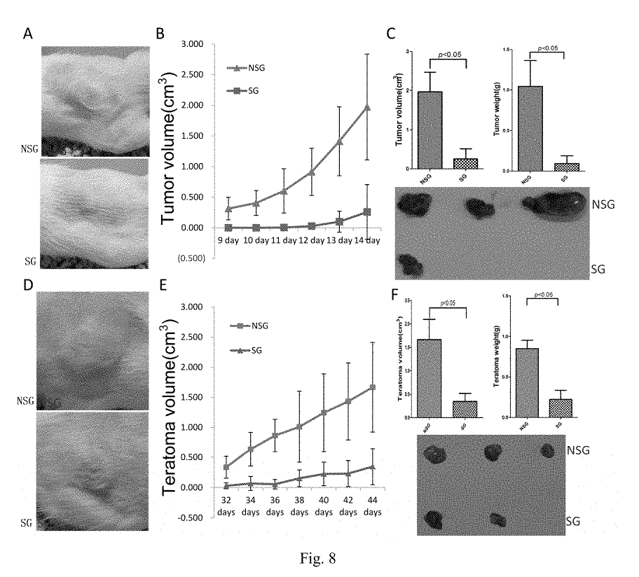

FIGS. 8A-8C show tumor formation situations (size, volume and growth situations) of lung cancer cells H460 subcutaneously in SG and NSG rats.

FIGS. 8D-8F show tumor formation situations (size, volume and growth situations) of human embryonic stem cells H9 subcutaneously in SG and NSG rats.

Finally, it should be noted that: the above embodiments are merely used to facilitate understanding of the technical solutions and core ideas of the present invention, and are not intended to limit the present invention. Although the present invention has been described in detail with reference to the foregoing embodiments, those skilled in the art should understand that the technical solutions described in the foregoing embodiments may be modified, or, part or all of the technical features may be equivalently substituted, and such modifications or substitutions are also within the protective scope of the claims of the present invention.

What is claimed is:

1. A construction method for an immunodeficient rat model, comprising the following steps:
(1) obtaining a gRNA targeted for knocking out a Prkdc gene of a rat DNA via in-vitro transcription and mixing the gRNA targeted for knocking out the Prkdc gene of the rat DNA with an in-vitro transcribed CAS9 mRNA, then freezing in RNase-free ultrapure water to obtain an injection A; obtaining a gRNA targeted for knocking out an IL2Rγ gene of the rat DNA via in-vitro transcription, and mixing the gRNA targeted for knocking out the IL2Rγ gene of the rat DNA with another in-vitro transcribed CAS9 mRNA, then freezing in RNase-free ultrapure water to obtain an injection B;
(2) amplifying and purifying a human SIRPα genomic DNA, and freezing in RNase-free ultrapure water to obtain an injection C;
(3) injecting the injection A and the injection B into cytoplasm of fertilized eggs of different rats by a microinjection method, and then transplanting the fertilized eggs into different pseudo-pregnant female rats, and breeding a second-generation rat A and a second-generation rat B;
(4) injecting the injection C into a pronucleus of a fertilized egg of a rat by microinjection method, and then transplanting the fertilized egg of the rat into a pseudo-pregnant female rat to obtain a second-generation rat C; and
(5) hybridizing the second-generation rat A, the second-generation rat B and the second-generation rat C to breed an F1 generation; further intercrossing the F1 generation to obtain IL2Rγ and Prkdc gene knockout immunodeficient rat models with human SIRPα gene.

2. The construction method for the immunodeficient rat model according to claim 1, wherein two gRNA sequences are used for knocking out the rat Prkdc gene in the step (1), which are respectively: the first exon gRNA sequence 1 as shown in SEQ ID NO: 8; the first exon gRNA sequence 2 as shown in SEQ ID NO: 9.

3. The construction method for the immunodeficient rat model according to claim 1, wherein two gRNA sequences are used for knocking out the rat IL2Rγ gene in the step (1), which are respectively: the second exon gRNA sequence 1 as shown in SEQ ID NO: 10; the fourth exon gRNA sequence 2 as shown in SEQ ID NO: 11.

4. The construction method for the immunodeficient rat model according to claim 3, wherein two other gRNA sequences are used for knocking out the rat IL2Rγ gene, which are respectively: the second exon gRNA sequence 1 as shown in SEQ ID NO: 10; the fourth exon gRNA sequence 3 as shown in SEQ ID NO: 12.

5. The construction method for the immunodeficient rat model according to claim 1, wherein in the step (1), an in-vitro transcription of the CAS9 mRNA is carried out by using a T7 transcription kit, a T3 transcription kit or an SP6 transcription kit.

6. The construction method for the immunodeficient rat model according to claim 1, wherein the pseudo-pregnant female rats in the step (3) and the step (4) are raised under SPF conditions.

7. The construction method for the immunodeficient rat model according to claim 1, wherein the fertilized eggs in the step (3) are taken from pregnant female rats raised under SPF conditions.

8. An immunodeficient rat model, wherein the immunodeficient rat model is an IL2Rγ and Prkdc gene knockout rat with human SIRPα gene.
